(19) United States
(12) Patent Application Publication
(10) Pub. No.: US 2024/0307334 A1
(43) Pub. Date: Sep. 19, 2024

(54) USE OF SOBETIROME AND PRODRUGS AND DERIVATIVES THEREOF FOR TREATING PITT-HOPKINS SYNDROME

(71) Applicants: Lieber Institute, Inc., Baltimore, MD (US); Oregon Health & Science University, Portland, OR (US)

(72) Inventors: Brady Maher, Baltimore, MD (US); Joseph Bohlen, Baltimore, MD (US); Thomas Scanlan, Portland, OR (US)

(21) Appl. No.: 18/407,998

(22) Filed: Jan. 9, 2024

Related U.S. Application Data
(60) Provisional application No. 63/479,084, filed on Jan. 9, 2023.

(51) Int. Cl. A61K 31/192; A61K 31/137; A61K 31/165; A61K 31/216; A61K 31/222; A61K 31/40; A61K 31/4025; A61K 31/439; A61K 31/445; A61K 31/4453; A61K 31/46; A61K 31/5386; A61P 25/28

(52) U.S. Cl. CPC A61K 31/192; A61K 31/137; A61K 31/165; A61K 31/216; A61K 31/222; A61K 31/40; A61K 31/4025; A61K 31/439; A61K 31/445; A61K 31/4453; A61K 31/46; A61K 31/5386; A61P 25/28

(57) ABSTRACT

The use of sobetirome or a prodrug or derivative thereof, including Sob-AM2, for treating a disease, disorder, or condition associated with or suspected of being associated with dysmyelination, such as Pitt-Hopkins Syndrome, is disclosed.

### FEDERALLY SPONSORED RESEARCH OR DEVELOPMENT

This invention was made with government support under grants MH110487 and DK052798 awarded by the National Institutes of Health. The government has certain rights in the invention.

### BACKGROUND

One form of syndromic autism spectrum disorder (ASD) is caused by autosomal dominant mutations in the transcription factor 4 (TCF4; not TCF7L2/T-Cell Factor 4) gene and results in Pitt-Hopkins syndrome (PTHS), a rare neurodevelopmental disorder characterized by intellectual disability, failure to acquire language, deficits in motor learning, hyperventilation, gastrointestinal abnormalities, and autistic behavior. Mouse models of PTHS consistently show behavioral deficits that approximate behavioral abnormalities observed in PTHS patients. The pathophysiological mechanisms underlying these behavioral deficits, however, are not completely understood.

### SUMMARY

In some aspects, the presently disclosed subject matter provides a method for treating a disease, disorder, or condition associated with or suspected of being associated with dysmyelination, the method comprising administering to a subject in need of treatment thereof a therapeutically effective amount of sobetirome or a prodrug or derivative thereof.

In certain aspects, the disease, disorder, or condition associated with or suspected of being associated with dysmyelination is selected from a neurodevelopmental disorder, an intellectual disability, an autism spectrum disorder, and combinations thereof.

In certain aspects, the administration of sobetirome or a prodrug or derivative thereof improves or attenuates the disease, disorder, or condition associated with or suspected of being associated with dysmyelination.

In particular aspects, the disease, disorder, or condition associated with or suspected of being associated with dysmyelination comprises Pitt-Hopkins Syndrome.

In certain aspects, the one or more conditions associated with Pitt-Hopkins Syndrome is selected from an intellectual disability, a developmental delay, breathing problems, recurrent seizures (epilepsy), delayed or lack of speech, impaired communication skills, impaired socialization skills, hyperventilation, apnea, cyanosis, constipation, gastrointestinal problems, microcephaly, myopia, strabismus, minor brain abnormalities, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems, and combinations thereof.

In certain aspects, the subject has one or more single nucleotide polymorphisms in a TCF4 gene. In certain aspects, the subject has a chromosomal deletion including at least a portion of a TCF4 gene. In certain aspects, the subject has a complete deletion of a TCF4 gene. In certain aspects, the subject has a chromosomal translocation comprising at least a portion of a TCF4 gene. In certain aspects, the subject has a translocation, frameshift, or non-sense mutation in a TCF4 gene.

In certain aspects, the subject is an infant or pediatric subject. In certain aspects, the subject has an age selected from about 16 years of age or less, about 12 years of age or less, about 8 years of age or less, about 5 years of age or less, and about 2 years of age or less. In certain aspects, the subject is an adult subject.

In some aspects, the administering of the sobetirome or a prodrug or derivative thereof is orally, parenterally, transdermally, sublingually, rectally, or intranasally. In certain aspects, the administration is by a single daily dose of sobetirome or a prodrug or derivative thereof. In certain aspects, the administration of sobetirome or a prodrug or derivative thereof is more than once daily.

In particular aspects, the prodrug of sobetirome comprises Sob-AM2.

In certain aspects, the method further comprises administering one or more therapeutic agents in combination with sobetirome or a prodrug or derivative thereof. In certain aspects, the one or more therapeutic agents are selected from clemastine, benzatropine, oxybutynin, trospium, ipratroprium, quetiapine, T3, XAV939, atropine, tiotropium, clobetasol, miconazole, hydroxyzine, oxiconazole, propafenone, benztropine, clotrimazole, tamoxifen, ketoconazole, dicyclomine, vesamicol, haloperidol, medroxyprogesterone, megestrol, ifenprodil, oxybutinin, bifonazole, cinanserin, betamethasone, methylprednisolone, econazole, and donepezil, or any other known or unknown pro-myelinating compound.

In certain aspects, the one or more therapeutic agents comprise a muscarinic receptor antagonist. In particular aspects, the muscarinic receptor antagonist is selected from atropine, glycopyrronium bromide, ipratropium bromide, oxybutynin, scopolamine, tiotropium bromide, benztropine, darifenacin, fesoterodine, trihexyphenidyl, tolterodine, trospium chloride, solifenacin, propantheline bromide, and propiverine, or any other known or unknown muscarinic receptor antagonist.

In other aspects, the presently disclosed subject matter provides for the use of sobetirome or a prodrug or derivative thereof the manufacture of a medicament for treating a disease, disorder, or condition associated with or suspected of being associated with dysmyelination, including Pitt-Hopkins Syndrome.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Drawings, which are not necessarily drawn to scale, and wherein:

FIG. 1A, FIG. 1B, FIG. 1C, and FIG. 1D demonstrate that clemastine increases maturation of oligodendrocytes (OLs) in Tcf4^{+/tr} in vitro cultures. (FIG. 1A) Graphical representation of plating and dosing regimen. (FIG. 1B) Immunocytochemistry of OPC/OL DIV14 in culture following 7 days of either vehicle (DMSO) or clemastine (1 M), cultures stained for DAPI (blue), PDGFRα (green), MBP (red) and Olig2 (gray). Scale bars represent 50 m. (FIG. 1C-FIG. 1D) Summary plots show Tcf4^{+/+} cells with vehicle (black) or treated with clemastine fumarate (gray) as well as Tcf4^{+/tr} cells with vehicle (dark blue) or treated with clemastine fumarate (sky blue). Representative results for response to vehicle and clemastine administration comparing PDGFRα/Olig2 (immature population) (Two-way ANOVA n=10-13 biologically independent animals/genotype F_{3,22}=14.39 p<0.0001 data are presented as mean values±SEM) and MBP+/Olig2+(Two-way ANOVA n=11-13 biologically independent animals/genotype F_{3,19}=5.966 p=0.0048 data are presented as mean values±SEM). These data show clemastine fumarate increased the percentage of mature OLs (MBP) while reducing the overall pool of OPCs (PDGFRα) in vitro. *p<0.05, **p<0.01, ***p<c0.001, ****p<0.0001

FIG. 2A, FIG. 2B, FIG. 2C, and FIG. 2D demonstrate that clemastine increases maturation of oligodendrocytes (OLs) in Tcf4^{+/tr} in vivo. (FIG. 2A) Graphical representation of the dosing regime in vivo. (FIG. 2B) Immunohistochemistry of OPC/OL in 55-μM tissue slices following 14 days of either vehicle (DMSO) or clemastine administration. Tissue stained for DAPI (blue), PDGFRα (green), MBP (red) and Olig2 (gray). Scale bars represent 50 m. (FIG. 2C-FIG. 2D) Summary plots show processed tissue from Tcf4^{+/+} mice with vehicle (black) or treated with clemastine fumarate (gray), as well as tissue from Tcf4^{+/tr} mice with vehicle (dark blue) or treated with clemastine fumarate (sky blue). Representative results for response to vehicle and clemastine administration comparing PDGFRα/Olig2 (immature population) (Two-way ANOVA n=10-13 biologically independent animals/genotype F_{3,23}=58.28 p<0.0001 data are presented as mean values±SEM) and CC-1/Olig2+(mature population) (Two-way ANOVA n=10-13 biologically independent animals/genotype F3,23=2620 p<0.0001 data are presented as mean values±SEM) These data show clemastine fumarate increased the percentage of mature OLs (MBP) while reducing the overall pool of OPCs (PDGFRα) in vivo. **p<0.05, **p<0.01, ***p<0.001, ****p<0.0001;

FIG. 3A, FIG. 3B, FIG. 3C, FIG. 3D, and FIG. 3E demonstrate that clemastine increases proportions of uncompacted myelin in Tcf4^{+/tr} mice. (FIG. 3A) Graphical representation of the dosing regime and subsequent TEM. (FIG. 313) Representative electron micrographs of the CC from vehicle/clemastine Tcf4^{+/+} and vehicle/clemastine Tcf4^{+/tr} mice. Arrows indicate compact and uncompact myelin designations. Scale bars represent 500 nm. (FIG. 3C) Density of myelination across all images for all conditions showing reduction of myelination in the vehicle and clemastine treated animals. (Two-way ANOVA biologically independent animals/genotype F=1.54, p=0.22) (FIG. 3D) Proportion of compact myelin per image showing less compaction overall in the clemastine Tcf4^{+/+} and clemastine Tcf4^{+/tr}. (Two-way ANOVA biologically independent animals/genotype F=1.24, p-value=0.26) (FIG. 3E) Proportion of uncompact myelin per image showing clemastine treatment increases uncompact myelin in both Tcf4^{+/+} and Tcf4^{+/tr} mice. (Two-way ANOVA biologically independent animals/genotype F=1.24 p-value=0.26) Center values represent the mean and error bars are S.E.M., *p<0.05, **p<0.01, ***p<0.001, ****p<0.0001;

FIG. 4A and FIG. 4B demonstrate that clemastine increases the number of pre-myelinating OLs in Tcf4^{+/tr} mice (FIG. 4A) Immunohistochemistry of OPC/OL in 55-μM tissue slices following 14 days of either vehicle (DMSO) or clemastine administration. Tissue stained for DAPI (blue), BCAS1 (red) and Olig2 (gray). Scale bars represent 50 μm. (FIG. 4B) Summary plots show processed tissue from Tcf4^{+/+} mice with vehicle (black) or treated with clemastine fumarate (gray), as well as tissue from Tcf4^{+/tr} mice with vehicle (dark blue) or treated with clemastine fumarate (sky blue). Representative results for response to vehicle and clemastine administration comparing BCAS1/Olig2 (pre-myelinating populations) (Two-way ANOVA n=6 biologically independent animals/genotype F_{1,5}=18.73 p<0.0001 data are presented as mean values±SEM). *p<0.05, **p<0.01, ***p<0.001, ****p<0.0001;

FIG. 5A, FIG. 5B, FIG. 5C, and FIG. 5D demonstrate that clemastine rescues pathological reduction of myelinated axons in Tcf4^{+/tr} mice. (FIG. 5A) Graphical representation of the dosing regime and subsequent electrophysiology. (FIG. 5B) Representative electrophysiology traces of evoked compound action potentials recorded in the CC from vehicle Tcf4^{+/tr} and clemastine treated Tcf4^{+/tr} mice. N1 represents action potentials traveling down myelinated axons and N2 represents action potentials traveling down unmyelinated axons. Example traces are normalized by the N1 peak. (FIG. 5C) The proportion of action potentials traveling down myelinated axons was consistently reduced in vehicle Tcf4^{+/tr} mice compared to clemastine treated Tcf4^{+/tr} mice. (p=0.034) (FIG. 5D) Linear regression fit of the N1/N2 ratio at its respective recording distance (ANCOVA F=2.72, p-value=0.019);

FIG. 6A, FIG. 6B, FIG. 6C, and FIG. 6D demonstrate that clemastine rescues behavioral deficits in Tcf4^{+/tr} mice. (FIG. 6A) Locomotor activity maps from Tcf4^{+/+} and Tcf4^{+/tr} mice untreated or treated for 14 days with clemastine fumarate. Movement was recorded for 30 min following placement in a 37.5 cm×37.5 cm novel open field arena. (FIG. 6B) Summary plots for distance traveled over 30 min depicts Tcf4^{+/+} mice untreated (black) or treated with clemastine fumarate (gray) as well as Tcf4^{+/tr} mice untreated (dark blue) or treated with clemastine fumarate (sky blue). These data show that Tcf4^{+/tr} mice treated with clemastine fumarate exhibited locomotor activity similar to treated Tcf4^{+/+} mice (n=8 biologically independent animals/genotype, F_{1,7}=20.41 p=0.0027, data are presented as mean values±SEM). (FIG. 6C and FIG. 6D) Summary plots for frequency of entering center (middle 15.25 cm×15.25 cm) show that Tcf4^{+/tr} mice treated with clemastine fumarate exhibited a. similar frequency of entering the center area compared to treated Tcf4^{+/+} mice (n=8 biologically independent animals/genotype, F_{1,7}=10.36 p=0.0147, data are presented as mean values±SEM). Asterisk (*) indicate the difference between mice groups (unpaired t-test). One symbol=p<0.05, two symbols=p<0.01, three symbols=p<0.001, four symbols=p<0.0001;

FIG. 7A, FIG. 713, FIG. 7C, FIG. 7D, FIG. 7E, and FIG. 7F demonstrate that sobetirome increases mature cells both in vitro and in vivo in Tcf4^{+/tr} mice. (FIG. 7A) Immunocytochemistry of OPC/OL DIV14 in culture following 7 days of either vehicle (DMSO) or sobetirome (1 μM), cultures stained for DAPI (blue), PDGFRα (green), MBP (red) and Olig2 (gray). Scale bars represent 50 μm. (FIG. 7B-FIG. 7C) Summary plots show Tcf4^{+/+} cells with vehicle (black) or treated with sobetirome (gray) as well as Tcf4^{+/tr} cells with vehicle (dark blue) or treated with sobetirome (sky blue). Representative results for response to vehicle and sobetirome administration comparing PDGFRα/Olig2 (immature population) (Two-way ANOVA n=8 biologically independent animals/genotype F_{1,7}=14.36 p=0.0068 data are presented as mean values±SEM) and MBP+/Olig2+(Two-way ANOVA n=8 biologically independent animals/genotype F_{1,6}=18.65 p=0.005 data are presented as mean values±SEM). These data show sobetirome increased the percentage of mature OLs (MBP) while reducing the overall pool of OPCs (PDGFRα) in vitro. (FIG. 7D) Immunohistochemistry of OPC/OL in 55-μM tissue slices following 14 days of either vehicle (DMSO) or Sob-AM2. Tissue stained for DAPI (blue), PDGFRα (green), CC-1 (red) and Olig2 (gray). (FIG. 7E-FIG. 7F) Summary plots show processed tissue from Tcf4^{+/+} mice with vehicle (black) or treated with Sob-AM2 (gray) as well as tissue from Tcf4^{+/tr} mice with vehicle (dark blue) or treated with Sob-AM2 (sky blue). (Two-way ANOVA n=4 biologically independent animals/genotype F_{1,3}=16.78 p=0.0263 data are presented as mean values±SEM) and CC-1+/Olig2+(Two-way ANOVA n=4 biologically independent animals/genotype F_{1,3}=15.57 p=0.029 data are presented as mean values±SEM). These data show sobetirome increased the percentage of mature OLs (MBP) while reducing the overall pool of OPCs (PDGFRα) in vivo;

FIG. 8A, FIG. 8B, FIG. 8C, and FIG. 8D demonstrate that Sob-AM2 rescues behavioral deficits in Tcf4^{+/tr} mice. (FIG. 8A) Locomotor activity maps from Tcf4^{+/+} and Tcf4^{+/tr} mice vehicle or treated for 14 days with Sob-AM2. Movement was recorded for 30 min following placement in a 37.5 cm×37.5 cm novel open field arena. (FIG. 8B) Summary plots for distance traveled over 30 min depicts Tcf4^{+/+} mice vehicle (black) or treated with Sob-AM2 (gray), as well as Tcf4^{+/tr} mice vehicle (dark blue) or treated with Sob-AM2 (sky blue). These data show that Tcf4^{+/tr} mice treated with Sob-AM2 exhibited locomotor activity similar to treated Tcf4^{+/+} mice, (n=8 biologically independent animals/genotype, F_{1,7}=19.95 p=0.0029 data are presented as mean values±SEM). (FIG. 8C and FIG. 8D) Summary plots for frequency of entering center (middle 15.25 cm×15.25 cm) show that Tcf4^{+/tr} mice treated with Sob-AM2 exhibited a similar frequency of entering the center area compared to treated Tcf4^{+/+} mice (n=8 biologically independent animals/genotype, T₁₄=0.2081, p>0.05, data are presented as mean values±SEM). Asterisk (*) indicate the difference between mice groups (unpaired t-test). One symbol=p<0.05, two symbols=p<0.01, three symbols=p<0.001, four symbols=p<0.0001;

FIG. 9A, FIG. 9B, FIG. 9C, and FIG. 9D demonstrate that clemastine has no effect on total number of oligodendrocytes; and

FIG. 10A, FIG. 10B, and FIG. 10C demonstrate that clemastine rescue remains effective even after halting treatment after p42 in Tcf4^{+/tr} mice (p90).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a method for treating a disease, disorder, or condition associated with or suspected of being associated with dysmyelination, the method comprising administering to a subject in need of treatment thereof a therapeutically effective amount of sobetirome or a prodrug or derivative thereof.

As used herein, sobetirome has the following chemical structure:

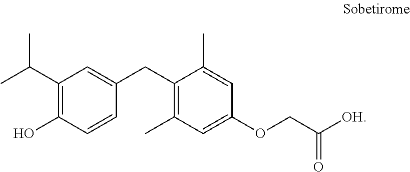 [SMILES: Cc1cc(OCC(=O)O)cc(C)c1Cc1ccc(O)c(C(C)C)c1]

In particular embodiments, the prodrug of sobetirome comprises Sob-AM2. Sob-AM2 has the following chemical structure:

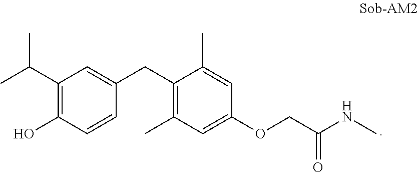 [SMILES: CNC(=O)COc1cc(C)c(Cc2ccc(O)c(C(C)C)c2)c(C)c1]

In some embodiments, the derivative or prodrug of sobetirome is one or more compounds disclosed in:
International PCT Patent Application Publication No. WO2016134292A1 for Derivatives of Sobetirome, to Scanlan et al., published Aug. 25, 2016;
U.S. Pat. No. 9,701,650 for Derivatives of Sobetirome, to Scanlan et al., issued Jun. 21, 2017;
U.S. Pat. No. 10,392,356 for Derivatives of Sobetirome, to Scanlan et al., issued Aug. 27, 2019;
U.S. Pat. No. 11,104,654 for Derivatives of Sobetirome, to Scanlan et al., issued Aug. 11, 2021;
International PCT Patent Application Publication No. WO2018032012A1 for Amide Compounds, Pharmaceutical Compositions Thereof, and Methods of Using the Same, to Scanlan et al., published Feb. 15, 2018; and
U.S. Pat. No. 11,325,886 for Amide Compounds, Pharmaceutical Compositions Thereof, and Methods of Using the Same, to Scanlan et al., issued Apr. 20, 2022, each of which is incorporated herein by reference in their entirety.

In certain embodiments, the disease, disorder, or condition associated with or suspected of being associated with dysmyelination is selected from a neurodevelopmental disorder, an intellectual disability, an autism spectrum disorder, and combinations thereof.

In certain embodiments, the administration of sobetirome or a prodrug or derivative thereof improves or attenuates the disease, disorder, or condition associated with or suspected of being associated with dysmyelination.

In particular embodiments, the disease, disorder, or condition associated with or suspected of being associated with dysmyelination comprises Pitt-Hopkins Syndrome.

In certain embodiments, the one or more conditions associated with Pitt-Hopkins Syndrome is selected from an intellectual disability, a developmental delay, breathing problems, recurrent seizures (epilepsy), delayed or lack of speech, impaired communication skills, impaired socialization skills, hyperventilation, apnea, cyanosis, constipation, gastrointestinal problems, microcephaly, myopia, strabismus, minor brain abnormalities, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems, and combinations thereof.

In certain embodiments, the subject has one or more single nucleotide polymorphisms in a TCF4 gene. In certain embodiments, the subject has a chromosomal deletion including at least a portion of a TCF4 gene. In certain embodiments, the subject has a complete deletion of a TCF4 gene. In certain embodiments, the subject has a chromosomal translocation comprising at least a portion of a TCF4 gene. In certain embodiments, the subject has a translocation, frameshift, or non-sense mutation in a TCF4 gene.

In certain embodiments, the subject is an infant or pediatric subject. In certain embodiments, the subject has an age selected from about 16 years of age or less, about 12 years of age or less, about 8 years of age or less, about 5 years of age or less, and about 2 years of age or less. In certain embodiments, the subject is an adult subject.

In some embodiments, the administering of the sobetirome or a prodrug or derivative thereof is orally, parenterally, transdermally, sublingually, rectally, or intranasally. In certain embodiments, the administration is by a single daily dose of sobetirome or a prodrug or derivative thereof. In certain embodiments, the administration of sobetirome or a prodrug or derivative thereof is more than once daily.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; poultry, such as domestic fowls including, but not limited to chickens, turkeys, geese, ducks, quail, guinea fowl, and pigeons; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

In certain embodiments, the presently disclosed method further comprises administering sobetirome and prodrugs and derivatives thereof, including Sob-AM2, in combination with one or more additional therapeutic agents.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In certain embodiments, the method further comprises administering one or more therapeutic agents in combination with sobetirome or a prodrug or derivative thereof. In certain embodiments, the one or more therapeutic agents are selected from clemastine, benzatropine, oxybutynin, trospium, ipratroprium, quetiapine, T3, XAV939, atropine, tiotropium, clobetasol, miconazole, hydroxyzine, oxiconazole, propafenone, benztropine, clotrimazole, tamoxifen, ketoconazole, dicyclomine, vesamicol, haloperidol, medroxyprogesterone, megestrol, ifenprodil, oxybutinin, bifonazole, cinanserin, betamethasone, methylprednisolone, econazole, and donepezil, or any other known or unknown pro-myelinating compound. These compounds have been shown to push OPCs to differentiate into OLs. See, for example, Mei et al., Nat Med 2014 and Najm et al., Nature 2015.

In certain embodiments, the one or more therapeutic agents comprise a muscarinic receptor antagonist. In particular embodiments, the muscarinic receptor antagonist is selected from atropine, glycopyrronium bromide, ipratropium bromide, oxybutynin, scopolamine, tiotropium bromide, benztropine, darifenacin, fesoterodine, trihexyphenidyl, tolterodine, trospium chloride, solifenacin, propantheline bromide, and propiverine, or any other known or unknown muscarinic receptor antagonist.

### C. Pharmaceutical Compositions and Administration

In another embodiment, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above.

In other embodiments, the presently disclosed subject matter provides for the use of sobetirome or a prodrug or derivative thereof the manufacture of a medicament for treating a disease, disorder, or condition associated with or suspected of being associated with dysmyelination, including Pitt-Hopkins Syndrome.

Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the presently disclosed compounds are administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. It has been found that the presently disclosed compounds are much more effective at penetrating the brain when administered intranasally.

Intranasal administration generally allows the active agent to bypass first pass metabolism, thereby enhancing the bioavailability of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the brain and/or peripheral nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™). Mixtures of mono-, di-, and triglycerides and mono- and di- fatty esters of PEG (Labrafil™), palmitate, glycerol monostearate, and phospholipids can be used to administer the presently disclosed compounds intranasally.

The presently disclosed compounds also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed compounds include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer, As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.).

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Clemastine Fumarate, Sobetirome, and Sob-AM2 Enhance Myelination and Promote Functional Recovery in a Syndromic ASD Mouse Model of Pitt-Hopkins Syndrome

### 1.1 Overview

Pitt-Hopkins syndrome (PTHS) is an autism spectrum disorder (ASD) caused by autosomal dominant mutations in the Transcription Factor 4 gene (TCF4). One pathobiological process caused by Tcf4 mutation is a cell autonomous reduction in oligodendrocytes (OLs) and myelination. In this study, we show that clemastine is effective at restoring myelination defects in a PTHS mouse model. In vitro, clemastine treatment reduced excess oligodendrocyte precursor cells (OPCs) and normalized OL density. In vivo, two-week intraperitoneal administration of clemastine also normalized OPC and OL density in the cortex of Tcf4 mutant mice and appeared to increase the number of axons undergoing myelination, as EM imaging of the corpus callosum showed a significant increase in uncompacted myelin. Importantly, this treatment paradigm resulted in functional rescue by improving electrophysiology and behavior. To confirm rescue was achieved via enhancing myelination, we show that treatment with sobetirome, a thyroid receptor analog, also is effective at normalizing OPC and OL densities and behavior in the PTHS mouse model. Together, these results provide preclinical evidence that pro-myelination therapies may be beneficial in PTHS and potentially other neurodevelopmental disorders characterized by dysmyelination.

### 1.2 Background

One form of syndromic autism spectrum disorder (ASD) caused by autosomal dominant mutations in the transcription factor 4 (TCF4; not TCF7L2/T-Cell Factor 4) gene results in Pitt-Hopkins syndrome (PTHS), a rare neurodevelopmental disorder characterized by intellectual disability, failure to acquire language, deficits in motor learning, hyperventilation, gastrointestinal abnormalities, and autistic behavior. Chen et al., 2021a. Mouse models of PTHS consistently show behavioral deficits that approximate behavioral abnormalities observed in PTHS patients. Thaxton et al., 2018; Cleary et al., 2021; Grubišič et al., 2015; Kennedy et al., 2016. The pathophysiological mechanisms underlying these behavioral deficits, however, are not completely understood.

Transcriptional profiling across 5 independent PTHS mouse models identified enrichment in differentially expressed genes (DEGs) related to myelination. This transcriptional profile was biologically validated, whereby in vivo, in vitro, and ex vivo experiments demonstrated mutations in TCF4 resulted in a reduction in oligodendrocytes (OLs) in conjunction with demyelination related functional deficits. Phan et al., 2020. Tcf4 is highly expressed in the entire OL lineage, and reductions in myelination due to Tcf4 mutations are cell autonomous. Phan et al., 2020; Kim et al., 2020; Marques et al., 2016; Wedel et al., 2020; Zhang et al., 2021.

Beyond PTHS, defects in the OL lineage are reported for a variety of ASD models and suggests this cell population may be a suitable target for therapeutic interventions. One well known pro-myelination compound is clemastine fumarate, which is a well-studied, FDA-approved, first-generation antihistamine predominantly used in the treatment of allergic conditions. Lee et al., 2021. It is a competitive antagonist at peripheral H1 receptors (H1R), blocking the actions of endogenous histamines, but its pro-myelination properties are thought to be through its muscarinic receptor 1 (M1R) activity. Mei et al., 2014; Chen et al., 2021b; Minigh, 2008. Several studies have consistently shown clemastine promotes differentiation of OPCs into mature/myelinating oligodendrocytes. Chen et al., 2021b; Li et al., 2015; Mei et al., 2016; Liu et al., 2016.

Another pro-myelination compound is sobetirome, a clinical-stage thyromimetic, that is effective at stimulating remyelination in several demyelination models. Hartley et al., 2019. In this study, we demonstrate that pharmacological enhancement of myelination with clemastine and sobetirome are a beneficial rescue approaches in a PTHS mouse model. We show administration of clemastine, both in vitro and in vivo, normalizes OPC and OL density, improves myelination, and normalizes electrophysiological and behavioral deficits. Similarly, we show that administration of sobetirome is effective both in vitro in vivo at normalizing OPC and OL density and also rescues behavioral deficits.

### 1.3 Methods

### 1.3.1 Animals and Tissue Collection

The Tcf4^{+/tr} mouse model of PTHS is heterozygous for an allele encoding deletion of the DNA-binding domain of TCF4 (B6; 129-TCF4tmlZhu/J, stock number 013598, Jackson Laboratory). This mouse colony was backcrossed for at least six generations, maintained by The Lieber Institute for Brain Developments Animal Facility on a 12-h light/dark cycle and fed ad libitum. Tcf4^{+/tr} mouse samples were matched with samples from Tcf4^{+/+} littermates, and sex was randomly selected in each genotype and age group. All procedures were performed in accordance with the National Institutes of Health Guide for the Care and Use of Laboratory Animals and approved by the Johns Hopkins University School of Medicine's Institutional Animal Care and Use Committee.

### 1.3.2 Clemastine Dosing Regime

A dosing solution at concentration 1 mg/mL was generated by weighing out the appropriate quantity of clemastine fumarate (Sigma SML0445) in a septa seal vial. 5% DMA (dimethylacetamide, Sigma 271012) was added up to the total volume needed. The clemastine fumarate was completely dissolved in the DMA prior to the addition of saline to the appropriate volume, Vehicle was consisting of 5% DMA in phosphate buffered saline. The mixture was shaken vigorously (vortex) and was ready for administration for intraperitoneal (1P) dosing at 10 mL/kg. The final pH was approximately 5. Animals were dosed every 24 hours for 14 consecutive days at a dose of 10 mg/kg for clemastine or an equal volume of vehicle depending on the condition. Initial dosing was performed at P28 up until P42 when animals were then used for subsequent experiments.

### 13.3 Sobetirome Dosing Regime

A dosing solution at concentration 1.0 mg/mL was generated by weighing out the appropriate quantity of sobetirome in a 15-mL falcon tube and resuspended in DMSO. 10 μM per day dose was used for in vitro studies (Sigma SML1900) following the previously described method of clemastine dosing in vitro. For in vivo IP injections we weighed out the appropriate quantity of Sob-AM2 (amide prodrug of sobetirome) and combined 1 mL of Kolliphor (C5135-5000), 1 mL of NMP (328634-100 mL) and 8 mL of Millipore water and warmed at 37° C. until a clear solution forms. Mice were dosed at 1.0 mg/kg/day for 14 days with either Sob-AM2 or NMP/Kolliphor solution (vehicle). Initial dosing was performed at P28 up until P42 when animals were then used for subsequent experiments.

1.3.4 Primary OPC and OL cultures

Primary OPC and OLs were obtained following a previous protocol¹⁹. In brief, P2-P3 pups were dissociated and plated at a density of 10.0×10³ cells/cm² in a 96 well ibidi plate coated in 0.1% polyethyleneimine (PEI) and 0.5-μg/mL laminin. Cells were plated and maintained in OPC proliferation media consisting of 1× StemPro Neural supplement (Thermo Fisher Scientific, A1050801), 1× Anti-Anti (Thermo Fisher Scientific, 15240-096), 10 ng/mL of Human FGF-basic (Peprotech, 100-18B) and 30 ng/mL rhPDGF-AA (R&D systems, 221-AA), with a half media exchange on DIV4. On DIV7 OL differentiation media was added (base media with removal of bFGF and rhPDGF-AA) and cells were differentiated into oligodendrocytes with the addition of either DMSO (Sigma D8418 at 0.1%) as a vehicle or Clemastine/Sobetirome (Sigma SML0445 10 μM/Sigma SML1900 10 μM) with media change every day until DIV14.

### 1.3.5 Immunohistochemistry and Immunocytochemistry

Cells were rinsed 1× with PBS and then fixed with 4% paraformaldehyde (PFA) for 5 minutes, following fixation cells were rinsed 3× with PBS. Similarly, mice were perfused with 20-30 mLs of 1× PBS, followed by 20-30 mLs of 4% PFA. Tissue was extracted and post fixed in 4% PFA on a rocker at 4° C. overnight. For immunostaining cells were rinsed 3× with 0.04% Tween 20 (Sigma 655204) while tissue (P42 mouse) was rinsed 3× with 0.4% Triton (Sigma X100). Cells were blocked in respective serum (10%) for 2 hours at room temperature on an orbital shaker. Following block, primary antibody was added in 2% serum in 0.04% Tween 20 for cells and 0.4% Triton-X 100 for tissue, and incubated overnight at 4° C. Following overnight incubation cells were rinsed 3× with 0.04% Tween 20 or 0.4% Triton respectively before adding the secondary antibody to incubate at room temperature for 2 hours. After incubation, cells were rinsed 3× in respective buffers and counterstained with DAPI (Invitrogen™, D1306). Visualization was carried out on a ZEISS LSM 700 Confocal. Imaging and quantification were performed blind to genotypes and conditions/treatments.

### 1.3.6 Transmission Electron Microscopy (TEM)

After perfusion with a 0.1 M sodium cacodylate buffer, pH 7.2, containing 2% paraformaldehyde (freshly prepared from EM grade aqueous solution), 2% glutaraldehyde, and 3 mM MgCl2, p42 mouse brains were kept overnight in fixative. The next day brains were dissected in fixative and rinsed with sodium cacodylate buffer. Samples were then post-fixed in reduced 2% osmium tetroxide, 1.6% potassium ferrocyanide in buffer (2 hr) on ice in the dark. Following a dH₂O rinse, samples were stained with 2% aqueous uranyl acetate (0.22 μm filtered, 1 hr, dark), dehydrated in a graded series of ethanol, propylene oxide and embedded in Eponate 12 (Ted Pella) resin. Samples were polymerized at 60° C. overnight. Thin sections, 60 to 90 nm, were cut with a diamond knife on the Reichert-Jung Ultracut E ultramicrotome and picked up with copper slot (1×2 mm) grids. Grids were stained with 2% uranyl acetate and observed with a Phillips CM120 TEM at 80 kV. Images were captured with an AMT XR80 CCD camera. Preparation of samples, TEM imaging, and quantification was performed blind to genotypes.

### 1.3.7 Electrophysiology

Acute coronal brain slices containing the corpus callosum (CC) were obtained from P39-P42 mice as previously described. Maher and LoTurco, 2012. Artificial cerebrospinal fluid (ACSF) was oxygenated (95% O₂ and 5% CO₂) and contained (in mM): 125 NaCl, 25 NaHCO₃, 1.25 NaH₂PO₄, 3 KCl, 25 dextrose, 1 MgCl₂, and 2 CaCl₂), pH 7.3. A bipolar stimulating electrode was placed 500 μm away from the midline and CC was stimulated with a 100 μs square pulse using 80% of the maximal stimulation intensity. The recording electrodes were fabricated from borosilicate glass (N51A, King Precision Glass, Inc.) to a resistance of 2-5 MΩ and placed at varying distances from the stimulating electrode in the contralateral CC. For cAP recording, pipettes were filled with ACSF. Voltage signals were recorded with an Axopatch 200B amplifier (Molecular Devices) and were filtered at 2 kHz using a built in Bessel filter and digitized at 10 kHz. Data was acquired using Axograph on a Dell PC. For electrophysiology experiments, data collection and analysis were performed blind to the conditions of the experiment.

### 1.3.8 Novel Open Field Assay

Locomotor activity and anxiety was assessed using Noldus PhenoTyper cages as previously shown. Mickelsen et al., 2019. Each cage is outfitted with two sets of cameras; one on the ceiling that faces the platform (35 cm×35 cm), and another pointed at the side of the cage. Mice were acclimated to the experimentation room in their home cages for at least 1 h. During acclimation, the Noldus EthoVision software was set-up to track movement for 30 total minutes. After acclimation, mice were placed in the center of the open field, opaque Plexiglas was placed on all four sides of the cage to obscure any visual cues, and the trial was started in the EthoVision software. After 30 min, the trial ended, and mice were placed back into their home cages. Noldus EthoVision software was used to determine distance traveled, time spent in center, and frequency of going to center.

### 1.3.9 Statistics

GraphPad Prism (GraphPad Software, San Diego, CA) was used to conduct statistical analyses for IHC, ICC, and behavioral experiments. Scipy stats package version 1.8.0 and Statsmodels package version 0.13.2 were used to conduct statistical analyses for EM and electrophysiology experiments. Data were analyzed using either a two-way analysis of variance (ANOVA), analysis of covariance (ANCOVA), or an unpaired t test. All ANOVA main effects were followed by Tukey post hoc tests.

### 1.4 Results

1.4.1 Clemastine induces maturation of OPCs in vitro

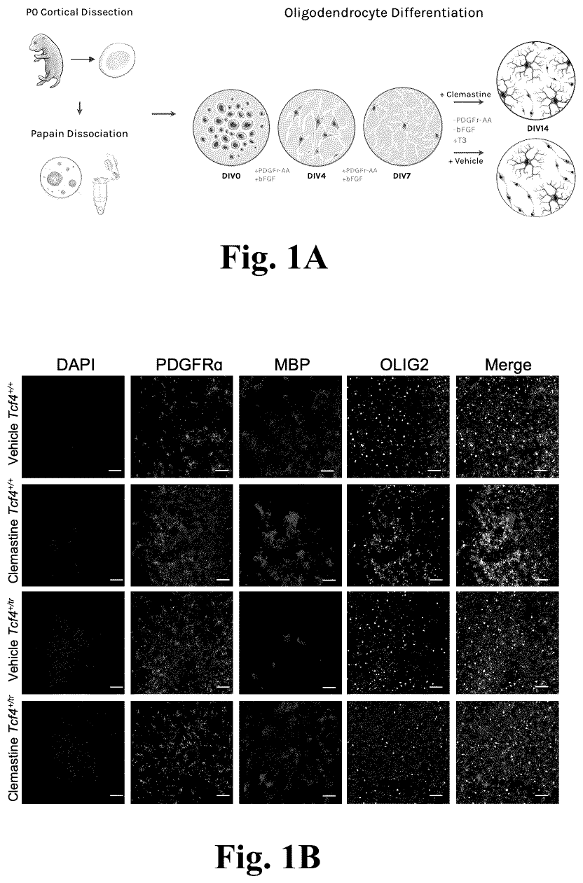

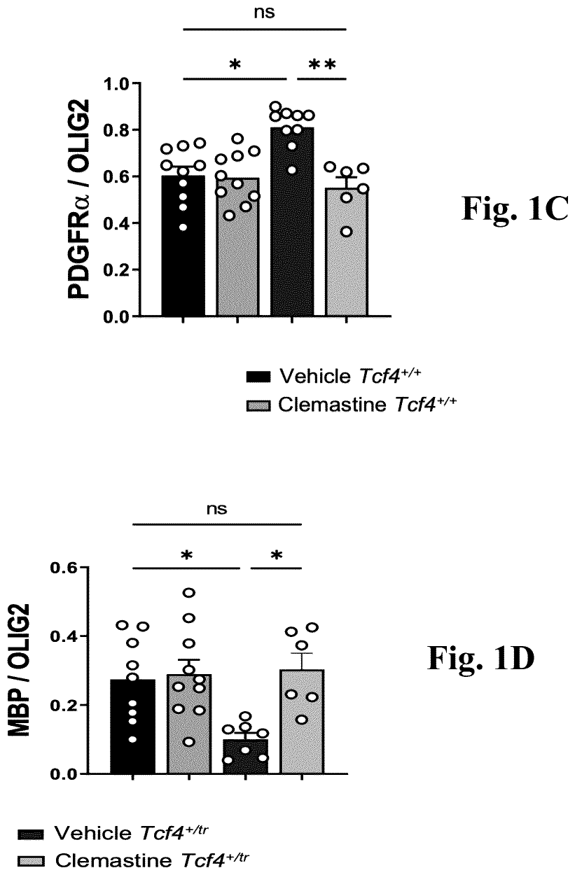

We first tested the effects of clemastine administration on maturation of OPCs in vitro. Previous studies indicate that clemastine is effective at promoting OPCs in primary cultures to differentiate into mature OLs (MBP+). Mei et al., 2014; Yoshida et al., 2020. Following a previously established protocol, Yoshida et al., 2020, we dissociated and plated OPCs from both Tcf4^{+/+} and Tcf4^{+/tr} mice onto 96-well plates in OPC proliferation media. OPCs were differentiated on DIV7 with OL differentiation media containing either clemastine (1 μM) or vehicle (DMSO) and fed every day with fresh media containing clemastine or vehicle before immunostaining on DIV14 (FIG. 1A). We performed blinded quantification of OPCs and OLs using antibodies against PDGFRα and MBP, respectively, and normalized our counts with the pan-OL marker OLIG2 (FIG. 1B). Consistent with previous observations, Phan et al., 2020, Tcf4^{+/tr} cultures treated with vehicle exhibited a significant decrease in mature OLs (MBP+/Olig2+) along with a significant increase in OPCs (PDGFRα+/Olig2+) when compared to littermate controls (FIG. 1C, FIG. 1D). Clemastine treatment resulted in a significant reduction in OPCs and an increase in OLs when compared to vehicle treated Tcf4^{+/tr} cells (FIG. 1C, FIG. 1D). These data indicate clemastine is effective at overcoming mutations in Tcf4 by promoting differentiation of OPCs into mature OLs and thereby normalizing the OL population to similar proportions of OPCs and OLs observed in Tcf4^{+/+} cultures.

### 1.4.2 Clemastine Increases the Number of CC-1 Positive Cells In Vivo

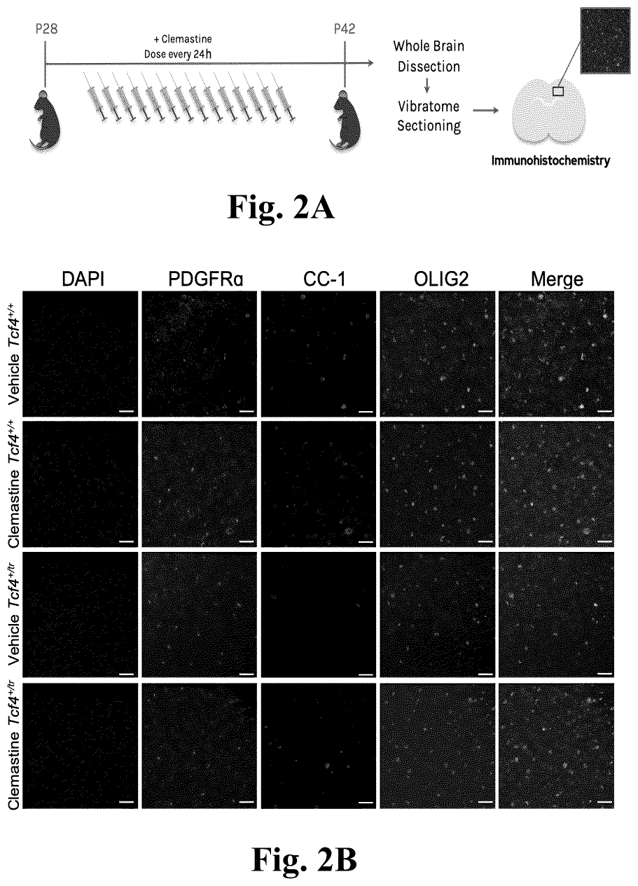

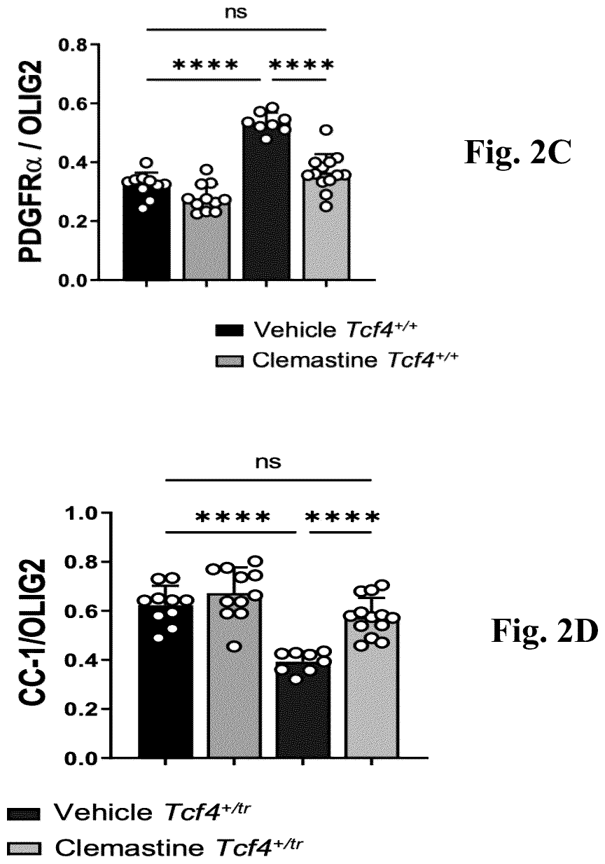

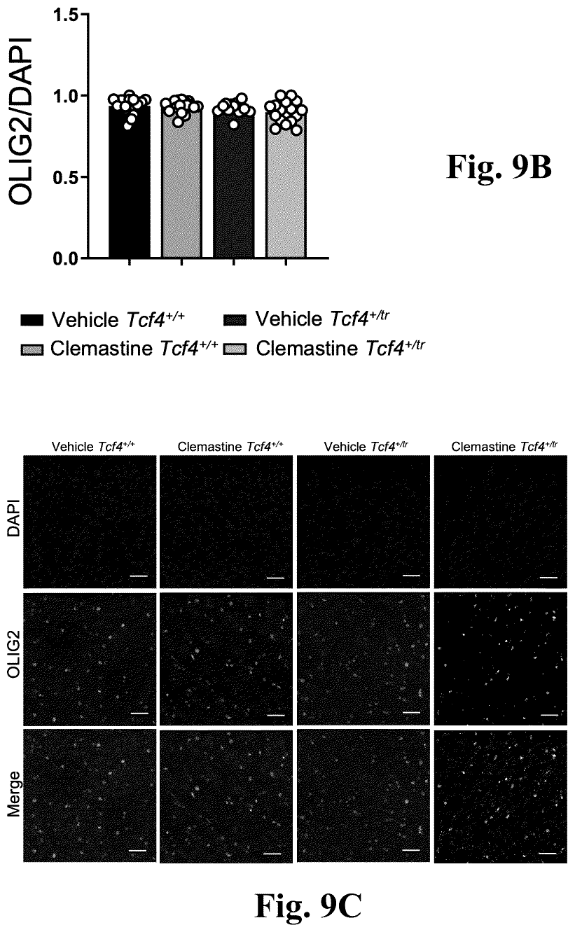

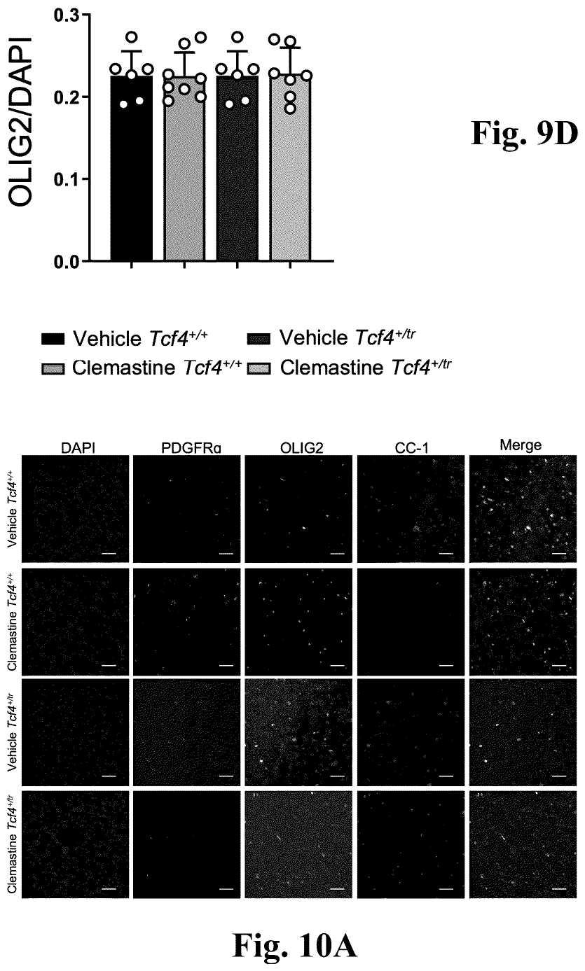

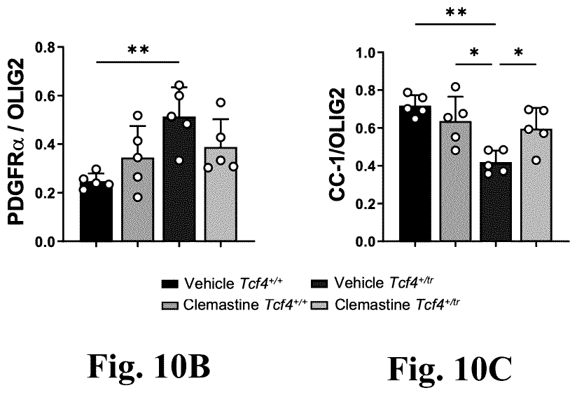

Next, we assessed the effectiveness of clemastine in vivo by dosing Tcf4^{+/tr} mice and Tcf4^{+/+} littermates intraperitoneally with either clemastine (10 mg/kg) or vehicle for two weeks followed by immunohistochemical (IHC) quantification (FIG. 2A). Consistent with prior results, Phan et al., 2020, vehicle-treated Tcf4^{+/tr} mice showed fewer mature OLs (CC1+/Olig2+) and more OPCs (PDGFRα+/Olig2+) when compared to vehicle-treated Tcf4^{+/+} littermates (FIG. 2C, FIG. 2D). Similar to our in vitro data, clemastine-treated Tcf4^{+/tr} mice showed a significant increase in mature OLs that coincided with a significant decrease in OPCs when compared to their vehicle-treated Tcf4^{+/+} littermates (FIG. 2C, FIG. 2D). Clemastine had no effect on the total number of Olig2 positive cells across all conditions for both in vitro and in vivo assays (FIG. 9). We also wanted to investigate the long term roll following clemastine administration. After dosing animals up to p42, we halted administration of clemastine and then collected samples at p90. Similar results were seen showing mature OLs are maintained after halting dosage (FIG. 10). Together, these data indicate clemastine also is effective in vivo at promoting OPCs to differentiate into OLs in a Tcf4^{+/tr} background, thereby normalizing the OL population to Tcf4^{+/+} levels.

### 1.4.3 Clemastine Increases Myelination Activity and Overall Myelination in Tcf4^{+/tr} Mice

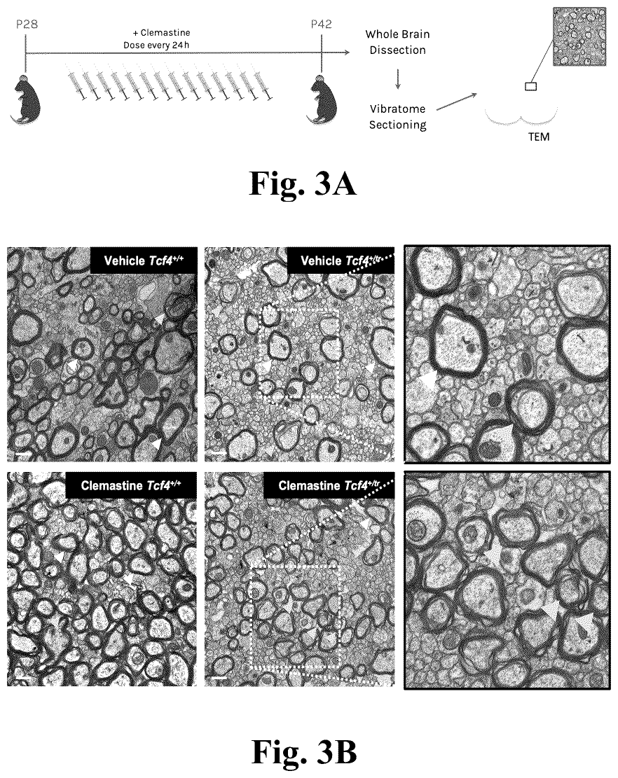

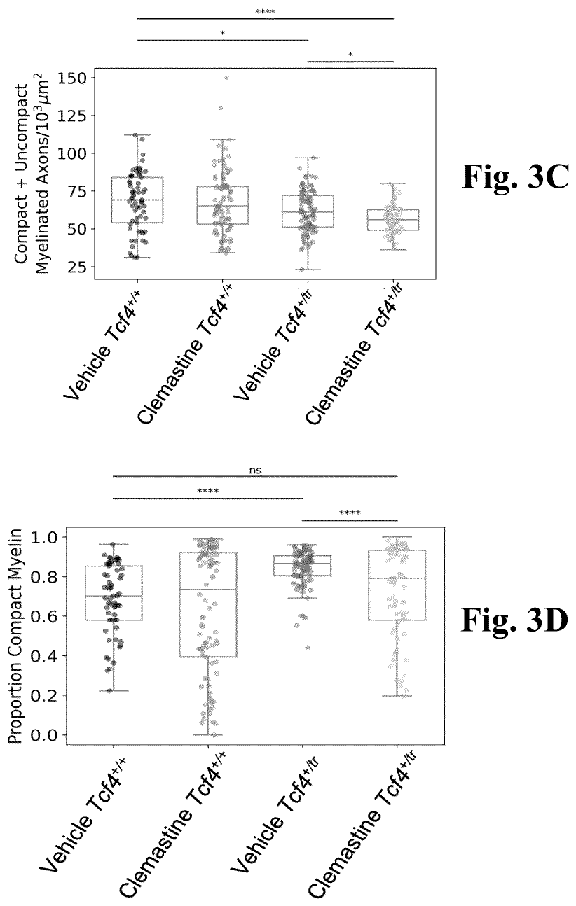

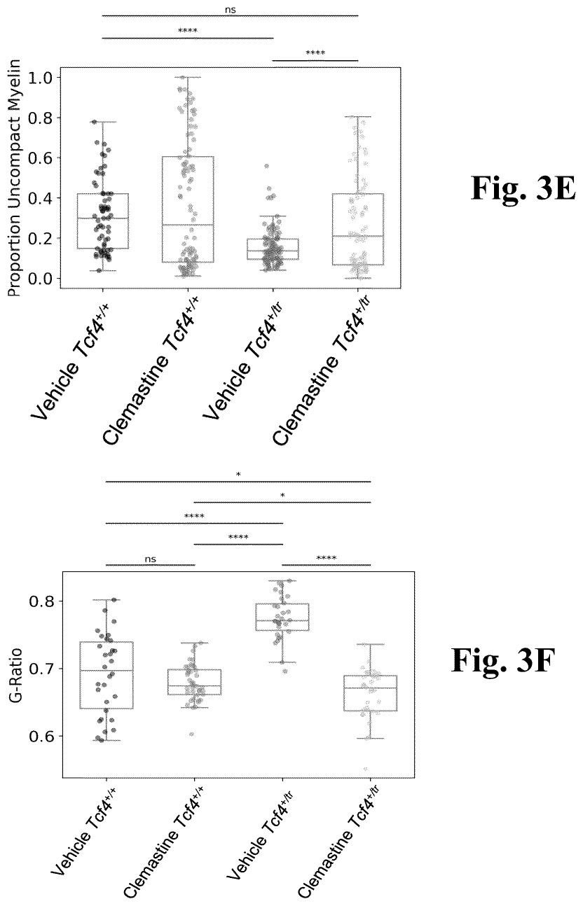

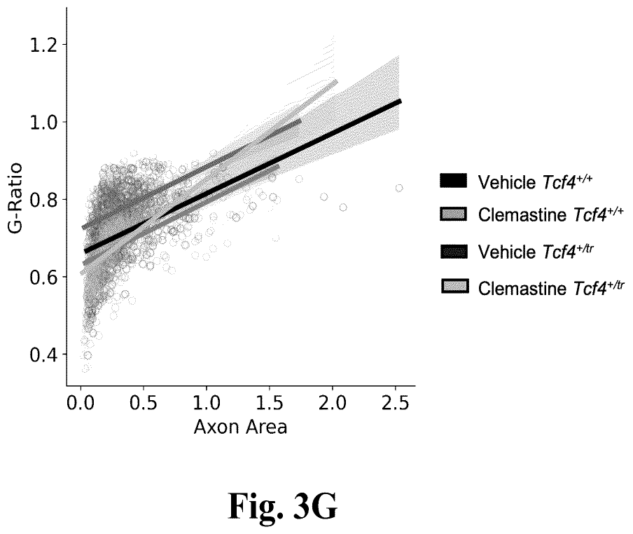

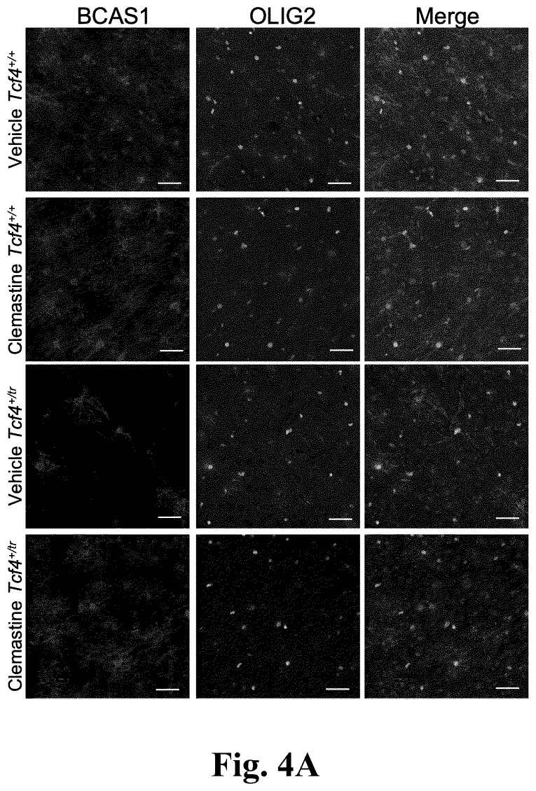

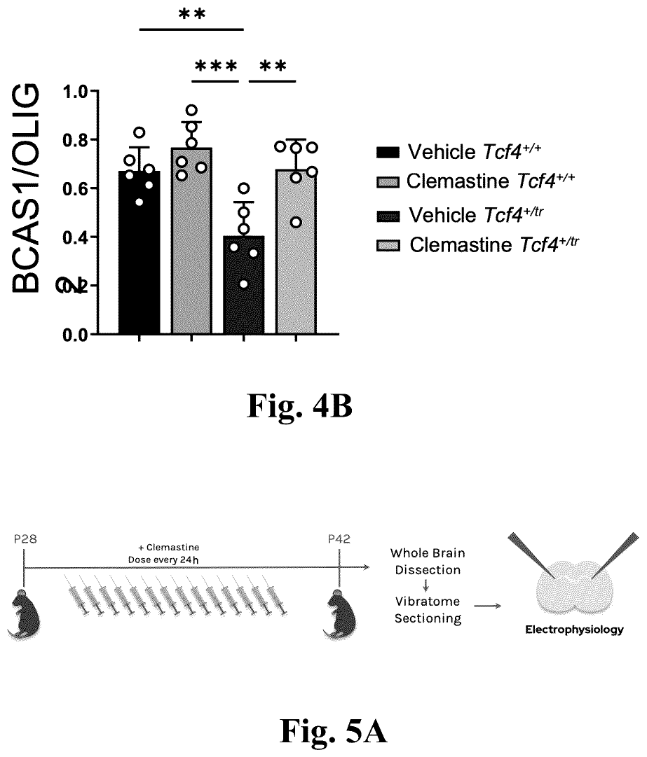

To visualize myelination, we used transmission electron microscopy (TEM) in both Tcf4^{+/+} and Tcf4^{+/tr} littermates that had been dosed with either vehicle or clemastine (FIG. 3A). Blinded to genotype and condition, TEM images were taken on the CC directly above the hippocampus from anatomically equivalent tissue sections in littermates. Following blinded quantification, we replicated our previous observation that Tcf4 mutation leads to a significant reduction in myelinated axons in the corpus callosum (FIG. 3B, FIG. 3C). We observed that the two week clemastine treatment, however, was unable to restore Tcf4^{+/+} levels of total myelin density in Tcf4^{+/tr} mice (FIG. 3B, FIG. 3C). We next classified myelinated axons as either compacted or uncompacted and observed that the balance between myelinated (compacted) and ongoing myelination (uncompacted) (FIG. 3B) was significantly different between vehicle-treated Tcf4^{+/tr} mice and vehicle-treated Tcf4^{+/+} littermates (FIG. 3D, FIG. 3E), suggesting that the active process of myelination was reduced in Tcf4^{+/tr} mice. Notably, we observed that clemastine treatment significantly increased the density of uncompacted myelin in Tcf4^{+/tr} mice compared to vehicle-treated Tcf4^{+/tr} mice, and normalized the proportion of uncompacted myelin to levels observed in vehicle-treated Tcf4^{+/+} mice (FIG. 3E), suggesting clemastine treatment is promoting the generation of new myelinated axons in the corpus callosum. To further validate this, we quantified BCAS1+ cells (pre-myelinating OLs). Similar to the EM data, clemastine treatment significantly increased the number of BCAS1+ cells in Tcf4^{+/tr} mice similar to Tcf4^{+/+} littermates (FIG. 4 A, FIG. 4B). Consistent with the in vivo and in vitro immunochemistry data, we see that clemastine is not only effective at enhancing the production of mature OLs (FIG. 1-FIG. 2), it also promotes the generation of new myelin ensheathment throughout this relatively short dosage time frame.

### 1.4.4 Clemastine Rescues Pathological Reduction of Myelinated Axons in Tcf4^{+/tr} Mice

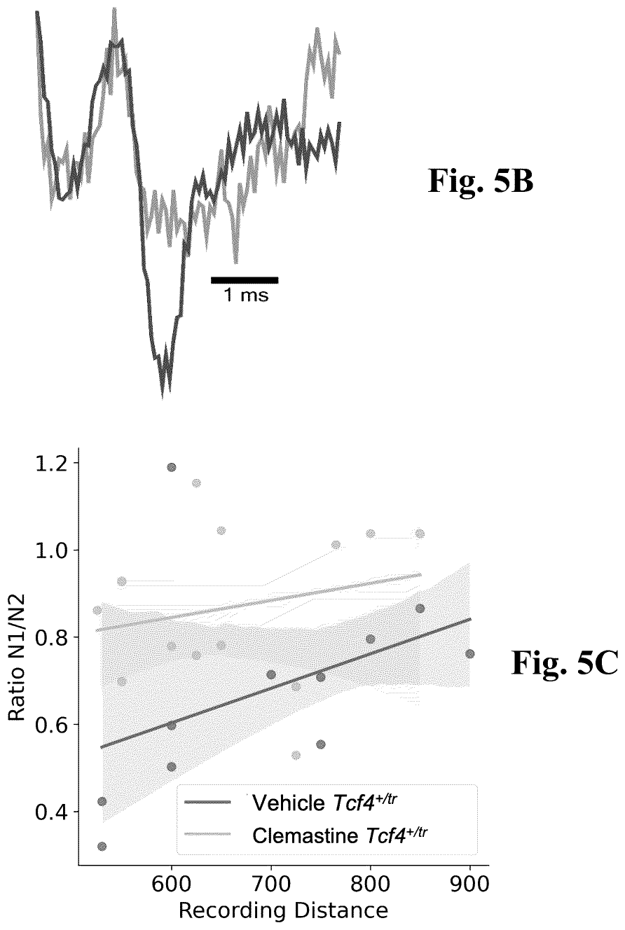

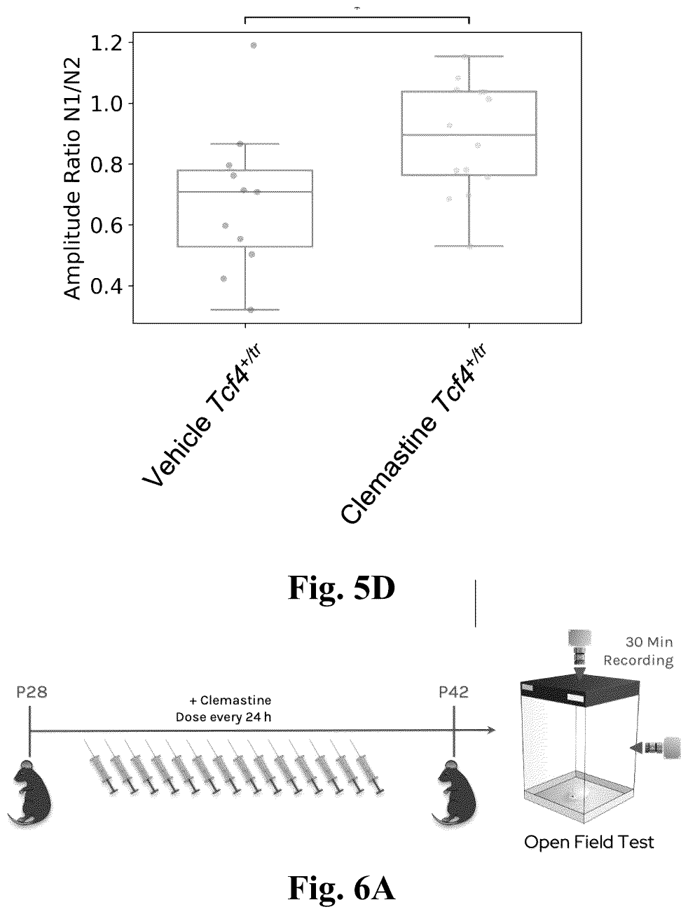

We next determined if clemastine's effect on the OL population was effective at normalizing physiology in Tcf4^{+/tr} mice. We measured the propagation of compound action potentials (CAPs) in the corpus callosum (CC) in acute brain slices from Tcf4^{+/+} and Tcf4^{+/tr} mice. CAPs were evoked by a bipolar stimulating electrode and recorded by a field electrode placed at varying distances across the CC and the amplitude of N1 and N2 peaks was quantified (FIG. 5A). The N1 and N2 peaks represent CAPs traveling down myelinated and unmyelinated axons, respectively, Olmos-Serrano et al., 2016, and we previously showed the N1/N2 ratio was significantly reduced in Tcf4^{+/tr} mice. Phan et al., 2020. Two-week clemastine treatment of Tcf4^{+/+} mice was effective at significantly increasing the N1/N2 ratio compared to vehicle-treated Tcf4^{+/tr} littermates (FIG. 5C). Our results indicate clemastine treatment is effective at improving the physiological function in the CC of the PTHS mouse model.

### 1.4.5 Clemastine Rescues Behavioral Deficits in Tcf4^{+/tr} Mice

Given the clemastine-dependent increases in the mature OL population and rescued electrophysiological in Tcf4^{+/tr} mice, we were interested to determine if these changes were adequate enough to ameliorate behavioral deficits in these mice. It was previously shown that a variety of PTHS mouse models display consistent behavioral deficits, including hyperlocomotion and reduced anxiety in the open field, among others. Thaxton et al., 2018; Kennedy et al., 2016; Ekins et al., 2020.

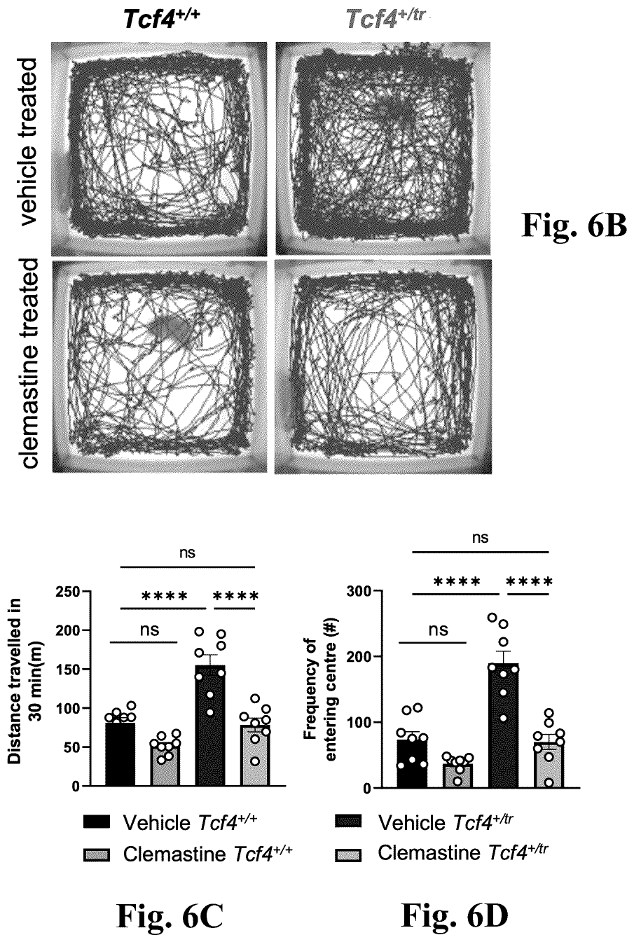

Therefore, we treated Tcf4^{+/tr} mice and Tcf4^{+/+} littermates with clemastine for 14 days and then assayed their behavior in the open field (FIG. 6A). Untreated Tcf4^{+/tr} animals exhibited an overall greater distance traveled and increased frequency of entering the center of the field (inversely related to anxiety) (FIG. 6B, FIG. 6C). Remarkably, clemastine-treated Tcf4^{+/tr} mice showed a significant reduction in total distance traveled and reduction in the frequency of entering the center of the field, which was similar to untreated Tcf4^{+/+} mice (FIG. 6B, FIG. 6C). These data indicate that clemastine treatment is effective at normalizing hyperlocomotion and anxiety phenotypes in the PTHS mouse model. All together, these data suggest pro-myelination therapy appears to be beneficial to cells, circuits, and behavior in a preclinical model of PTHS and supports the notion that clemastine or other pro-myelination agents could be an applicable therapeutic intervention in humans diagnosed with PTHS.

### 14.6 Sobetirome Rescues In Vitro, In Vivo and Behavior in Tcf4^{+/tr} Mice

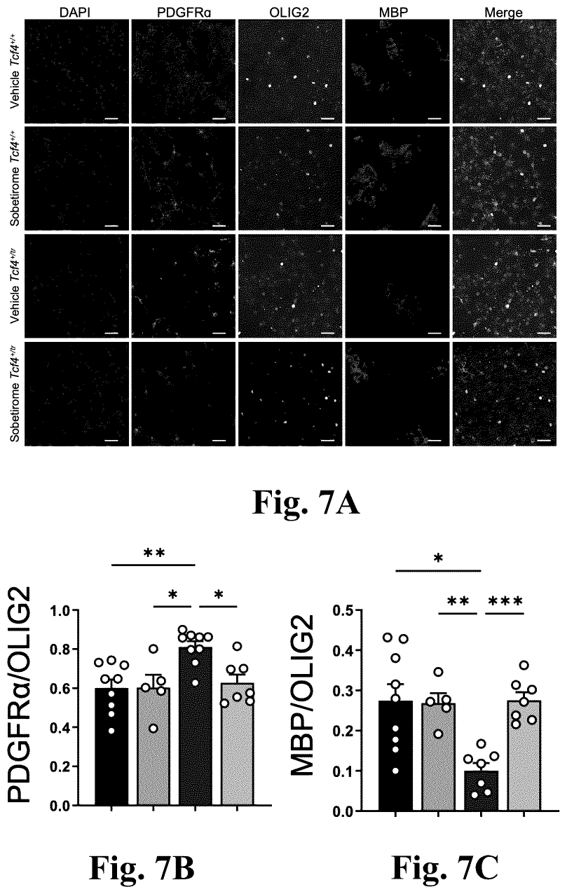

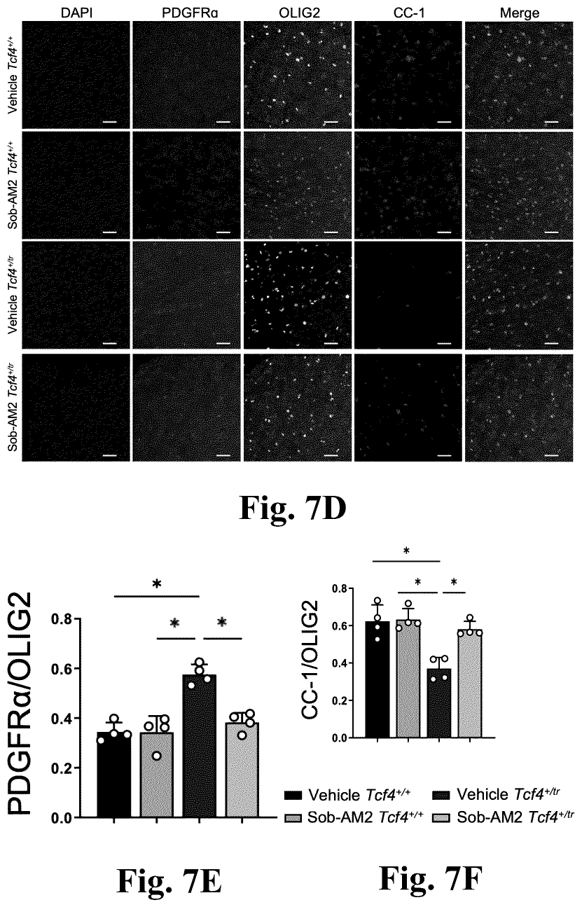

The polypharmacology of clemastine makes it difficult to ascertain if its behavioral normalization in the PTHS mouse model results from its pro-myelination capabilities or through its direct actions on the M1 and/or H1 receptor. Therefore, to discriminate between clemastine's two potential mechanisms of action, we tested the efficacy of sobetirome, a thyroid hormone agonist, and a CNS-selective prodrug of sobetirome, Sob-AM2, which were previously shown to promote OPC differentiation and remyelination in preclinical mouse models of demyelination. Hartley et al., 2019. We performed blinded quantification of OPCs and OLs using antibodies against PDGFRα and MBP, respectively, and normalized our counts with the pan-OL marker OLIG2 (FIG. 7A). Consistent with previous observations, Phan et al., 2020, Tcf4^{+/tr} cultures treated with vehicle exhibited a significant decrease in mature OLs (MBP+/Olig2+) along with a significant increase in OPCs (PDGFRα+/Olig2+) when compared to littermate controls (FIG. 7B, FIG. 7C).

Sobetirome treatment resulted in a significant reduction in OPCs and an increase in OLs when compared to vehicle-treated Tcf4^{+/tr} cells (FIG. 7B, FIG. 7C). These data indicate sobetirome is effective at overcoming the deleterious effects of Tcf4 mutation on OL development by promoting differentiation of OPCs into mature OLs, and thereby normalizing the OL population to similar proportions of OPCs and OLs as observed in Tcf4^{+/+} cultures.

Next, we assessed the effectiveness of Sob-AM2 in vivo by dosing Tcf4^{+/tr} mice and Tcf4^{+/+} littermates intraperitoneally with either Sob-AM2 (1 mg/kg) or vehicle for two weeks followed by immunohistochemical (IHC) quantification (FIG. 7D). Consistent with prior results, Phan et al., 2020, vehicle-treated Tcf4^{+/tr} mice showed fewer mature OLs (CC1+/Olig2+) and more OPCs (PDGFRα+/Olig2+) when compared to vehicle-treated Tcf4^{+/+} littermates (FIG. 7E, FIG. 7F). Similar to our in vitro data, Sob-AM2-treated Tcf4^{+/tr} mice showed a significant increase in mature OLs that coincided with a significant decrease in OPCs when compared to vehicle-treated Tcf4^{+/+} littermates (FIG. 7E, FIG. 7F). Together, these data indicate sobetirome and Sob-AM2 are effective both in vitro and in vivo at promoting OPCs to differentiate into OLs in a mutant Tcf4 background, thereby normalizing the OL population to Tcf4^{+/+} levels.

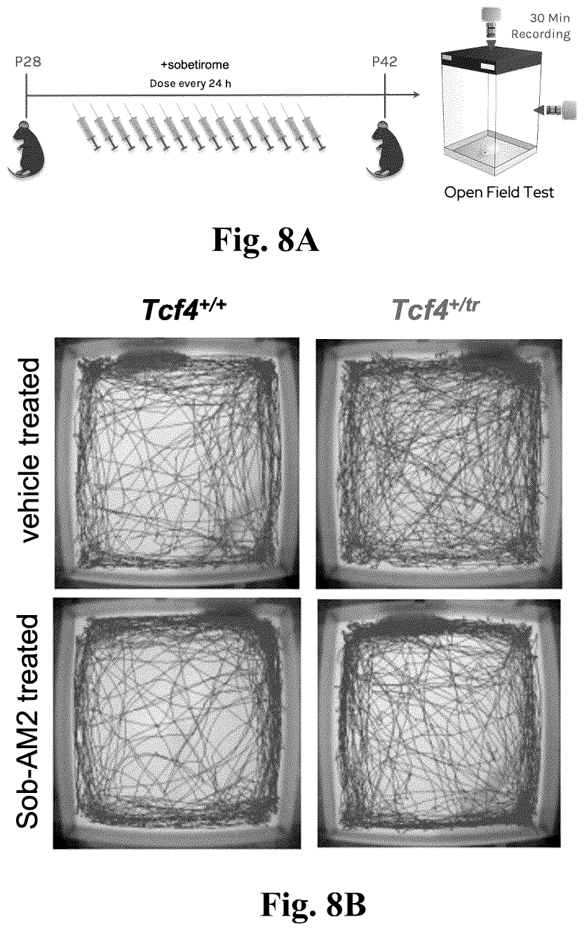

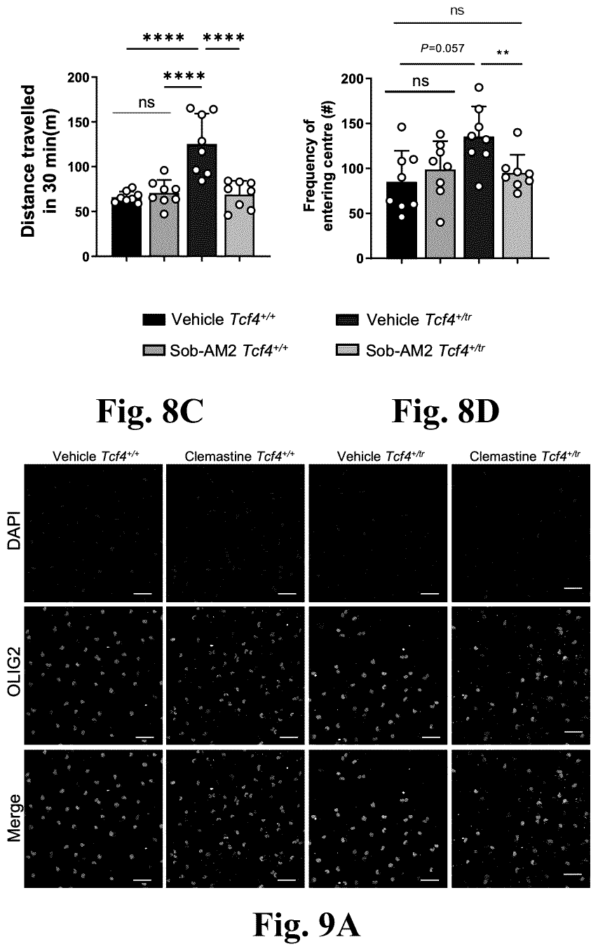

Next we treated Tcf4^{+/tr} mice and Tcf4^{+/+} littermates with Sob-AM2 for 14 days and then assayed their behavior in the open field (FIG. 8A). Vehicle Tcf4^{+/tr} animals exhibited an overall greater distance traveled and a modest increased frequency of entering the center of the field (inversely related to anxiety) (FIG. 8B, FIG. 8C). Remarkably, Sob-AM2-treated Tcf4^{+/tr} mice showed a significant reduction in total distance traveled (FIG. 8C) and reduction in the frequency of entering the center of the open field (FIG. 81D) compared to vehicle-treated Tcf4^{+/tr} littermates.

These data indicate that Sob-AM2 treatment is effective at normalizing hyperlocomotion and anxiety phenotypes in the PTHS mouse model. All together, these data suggest pro-myelination therapy appears to be beneficial to cells, circuits, and behavior in a preclinical model of PTHS and supports the notion that pro-myelination agents could be an applicable therapeutic intervention in humans diagnosed with PTHS.

### 1.5 Discussion

We have performed a series of experiments to demonstrate that pharmacological enhancement of myelination is effective at normalizing abnormal brain function and behavior in a mouse model of PTHS. We demonstrate that clemastine, a previously described remyelination compound, is effective at normalizing OPC and OL density both in vitro and in vivo (FIG. 1, FIG. 2). Additionally, EM analysis showed that clemastine treatment elevated the levels of uncompacted myelin indicative of ongoing myelination (FIG. 3). BCAS1+ staining further validated the increase in these pre-myelinating cells in clemastine treated animals (FIG. 4). This improved OL maturation resulted in functional rescue, whereby more CAPs were observed traveling down myelinated axons following treatment with clemastine (FIG. 5). Lastly, we showed that the clemastine-dependent improvement of OL populations and physiological function normalized behavioral deficits (FIG. 6). To assess if clemastine recovery was due to pro-myelination or other targets (H1R and/or M1R), we repeated the in vitro and in vivo studies with sobetirome and Sob-AM2, which induce OPC differentiation through thyroid receptor activation. Hartley et al., 2019. Sobetirome not only increased mature OLs in vitro and in vivo (FIG. 7), but also normalized behavioral deficits to a similar degree as clemastine (FIG. 8). Together, these results provide strong evidence that pharmacological enhancement of myelination is responsible for behavioral rescue in the PTHS mouse model and therefore identifies a potential therapeutic approach for PTHS.

### 1.5.1 Myelination Deficits in PTHS

Myelination deficits are consistently observed in several mouse models of PTHS. The initial observation was derived from transcriptomic analysis of five different PTHS mouse models and was subsequently biologically confirmed in the same Tcf4^{+/tr} mouse model used in this study. Phan et al., 2020. Regulation of oligodendrocyte development by Tcf4 was also shown in the mouse spinal cord where homozygous knockout of the long isoform of Tcf4 resulted in a significant reduction in mature OLs. Wedel et al., 2020. Moreover, conditional deletion of Tcf4 in the Nkx2.1 lineage resulted in a significant increase in OPCs of the mouse olfactory bulb. Zhang et al., 2021.

Tcf4 is expressed in all stages of OL development as demonstrated by single cell sequencing and fluorescent in situ hybridization, and a cell autonomous effect of Tcf4 on OL phenotypes is established. Phan et al., 2020; Marques et al., 2016; Wedel et al., 2020; Zhang et al., 2021. Clinical evidence for myelination deficits in PTHS patients is currently qualitative, due to the rare occurrence of this syndrome, with reports of several patients showing delayed myelination, white matter hyperintensities, and dysplasia of the corpus callosum. Amiel et al., 2007; Goodspeed et al., 2018; Rosenfeld et al., 2009; Brockschmidt et al., 2011; Stavropoulos et al., 2010. Future studies using PTHS patient-derived induced pluripotent stem cells will be important to demonstrate OL phenotypes observed in the mouse model translate to the human condition.

### 1.5.2 Therapeutic Potential of Clemastine and Sobetirome

There are currently no clinically approved therapies that promote myelination. (Hartley et al. 2014; Plemel et al. 2017). The pro-myelinating capabilities of clemastine were first identified in a drug screen that was specific for functional myelination. Mei et al., 2014. Clemastine is an FDA-approved, first generation antihistamine, that readily crosses the blood brain barrier, however its effect on OPCs appears to be through its off-target antimuscarinic effects. Mei et al., 2014; Mei et al., 2016. Clemastine administration has rescued myelination defects in a variety of mouse models of demyelination, neurodegeneration, and a neurodevelopmental disorder. Lee et al., 2021; Li et al., 2015; Mei et al., 2016; Liu et al., 2016; Xie et al., 2021; Barak et al., 2019; Deshmukh et al., 2013; Cree et al., 2018. Moreover, it showed remyelination capabilities in a phase 2 clinical trial for multiple sclerosis. Green et al., 2017.

Thyroid hormone is a critical signaling molecule that regulates the proliferation and differentiation of OPCs and subsequent development of myelination through its activation of TRs found in OPCs. Barres et al., 1994; Gao et al., 1998; Billon et al., 2002.

Sobetirome is a clinical-stage TR agonist that is shown to promote myelin repair in clinical models of demyelination 18, 30. Hartley et al., 2019; Hartley et al., 2014. Importantly, sobetirome does not show any adverse side effects associated with excess thyroid hormone because of its unique tissue distribution and specificity for TRbeta over TRalpha receptors. Scanlan, 2010; Ferrara et al., 2017; Placzek et al., 2016. The sobetirome prodrug Sob-AM2 is better able to cross the blood brain barrier and distribute to the central nervous system following systemic administration, making it suitable for clinical evaluation in demyelinating diseases. Placzek et al., 2016; Manzano et al., 2003.

Here, we demonstrate that both clemastine and sobetirome/Sob-AM2 are effective at rescuing myelination deficits and behavior in a mouse model of PTHS. Consistent with previous findings, our results suggest the cellular mechanism for the effect of these compounds is through promoting OPCs to differentiate, as we showed that both compounds shift the OL population by reducing OPCs and increasing OLs (FIG. 1, FIG. 2, and FIG. 7). Clemastine is a H1R antagonist and its effect on OPC differentiation is proposed to work through M1R antagonism, Mei et al., 2014; Chen et al., 2021; Minigh et al., 2008, whereas sobetirome promotes myelination through activation of TRs. We reason that because these two compounds promote myelination through two different modes of action (i.e., M1R or TR), it therefore indicates the mechanisms underlying behavioral rescue is through their common ability to promote myelination. Moreover, these results also suggest that any pro-myelinating compound will likely be effective at normalizing behavior in a PTHS mouse model, and therefore greatly improves the chance of developing a pro-myelinating therapeutic that will be efficacious and compatible with PTHS patients.

TABLE 1
List of Antibodies

| | | | Catalog |
|---|---|---|---|
| Antibody | Dilution | Company | No. |
| Rabbit anti-Olig2 | 1:500 | EMD Millipore | AB9610 |
| Mouse anti-APC (CC-1) | 1:500 | Calbiochem | OP80- |
| | | | 100UG |
| Rat anti-PDGFRα | 1:500 | BD Pharmingen | 558774 |
| Mouse anti-MBP (F-6) | 1:500 | Santa Cruz | Sc-271524 |
| | | Biotechnology | |
| Rat anti-MBP (aa82-87) | 1:50 | Bio Rad | MCA409S |
| Rabbit Bcas1 | 1:500 | Synaptic Systems | 445003 |
| Alexa Fluor 546 Goat anti- | 1:1000 | Invitrogen | A11010 |
| Rabbit | | | |
| Alexa Fluor 488 Goat anti-Rat | 1:1000 | Invitrogen | A48262 |
| Alexa Fluor 647 Donkey anti- | 1:1000 | Invitrogen | A31571 |
| Mouse | | | |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Chen H-Y, Bohlen J F, Maher B J. Molecular and Cellular Function of Transcription Factor 4 in Pitt-Hopkins Syndrome. Dev Neurosci. 2021a; 43(3-4):159-167.
Thaxton C, Kloth A D, Clark E P, Moy S S, Chitwood R A, Philpot B D. Common Pathophysiology in Multiple Mouse Models of Pitt-Hopkins Syndrome. J Neurosci. 2018; 38(4):918-936.
Cleary C M, James S, Maher B J, Mulkey D K. Disordered breathing in a Pitt-Hopkins syndrome model involves Phox2b-expressing parafacial neurons and aberrant Nav1.8 expression. Nat Commun. 2021; 12(1):5962.
Grubišič V, Kennedy A J, Sweatt J D, Parpura V. Pitt-Hopkins Mouse Model has Altered Particular Gastrointestinal Transits In Vivo. Autism Res. 2015; 8(5):629-633.
Kennedy A J, Rahn E J, Paulukaitis B S, et al. Tcf4 regulates synaptic plasticity, DNA methylation, and memory function. Cell Rep. 2016; 16(10):2666-2685.
Phan B N, Bohlen J F, Davis B A, et al. A myelin-related transcriptomic profile is shared by Pitt-Hopkins syndrome models and human autism spectrum disorder. Nat Neurosci. 2020; 23(3):375-385.
Kim H, Berens N C, Ochandarena N E, Philpot B D. Region and cell type distribution of TCF4 in the postnatal mouse brain. Front Neuroanat. 2020; 14:42.
Marques S, Zeisel A, Codeluppi S, et al. Oligodendrocyte heterogeneity in the mouse juvenile and adult central nervous system. Science. 2016; 352(6291):1326-1329.
Wedel M, Frob F, Elsesser 0, et al. Transcription factor Tcf4 is the preferred heterodimerization partner for Olig2 in oligodendrocytes and required for differentiation. Nucleic Acids Res. 2020; 48(9):4839-4857.
Zhang X, Huang N, Xiao L, Wang F, Li T. Replenishing the aged brains: targeting oligodendrocytes and myelination? Front Aging Neurosci. 2021; 13:760200.
Lee J I, Park J W, Lee K J, Lee D H. Clemastine improves electrophysiologic and histomorphometric changes through promoting myelin repair in a murine model of compression neuropathy. Sci Rep. 2021; 11(1):20886.
Mei F, Fancy SPJ, Shen Y-A A, et al. Micropillar arrays as a high-throughput screening platform for therapeutics in multiple sclerosis. Nat Med. 2014; 20(8):954-960.
Chen J-F, Liu K, Hu B, et al. Enhancing myelin renewal reverses cognitive dysfunction in a murine model of Alzheimer's disease. Neuron. 2021b; 109(14):2292-2307.e5.
Minigh J. Clemastine. In: XPharm: The Comprehensive Pharmacology Reference. Elsevier; 2008:1-6.
Li Z, He Y, Fan S, Sun B. Clemastine rescues behavioral changes and enhances remyelination in the cuprizone mouse model of demyelination. Neurosci Bull. 2015; 31(5):617-625.
Mei F, Lehmann-Horn K, Shen Y-A A, et al. Accelerated remyelination during inflammatory demyelination prevents axonal loss and improves functional recovery. eLife. 2016; 5.
Liu J, Dupree J L, Gacias M, et al. Clemastine enhances myelination in the prefrontal cortex and rescues behavioral changes in socially isolated mice. J Neurosci. 2016; 36(3):957-962.
Yoshida A, Takashima K, Shimonaga T, Kadokura M, Nagase S, Koda S. Establishment of a simple one-step method for oligodendrocyte progenitor cell preparation from rodent brains. J Neurosci Methods. 2020; 342:108798.
Maher B J, LoTurco J J. Disrupted-in-schizophrenia (DISC1) functions presynaptically at glutamatergic synapses. PLoS ONE. 2012; 7 (3):e34053.
Mickelsen L E, Bolisetty M, Chimileski B R, et al. Single-cell transcriptomic analysis of the lateral hypothalamic area reveals molecularly distinct populations of inhibitory and excitatory neurons. Nat Neurosci. 2019; 22(4):642-656.
Olmos-Serrano J L, Kang H J, Tyler W A, et al. Down syndrome developmental brain transcriptome reveals defective oligodendrocyte differentiation and myelination. Neuron. 2016; 89(6):1208-1222.
Ekins S, Puhl A C, Davidow A. Repurposing the dihydropyridine calcium channel inhibitor nicardipine as a nav1.8 inhibitor in vivo for Pitt Hopkins Syndrome. Pharm Res. 2020; 37(7):127.
Amiel J, Rio M, de Pontual L, et al. Mutations in TCF4, encoding a class I basic helix-loop-helix transcription factor, are responsible for Pitt-Hopkins syndrome, a severe epileptic encephalopathy associated with autonomic dysfunction. Am J Hum Genet. 2007; 80(5):988-993.
Goodspeed K, Newsom C, Morris M A, Powell C, Evans P, Golla S. Pitt-Hopkins Syndrome: A Review of Current Literature, Clinical Approach, and 23-Patient Case Series. J Child Neurol. 2018; 33(3):233-244.
Rosenfeld J A, Leppig K, Ballif B C, et al. Genotype-phenotype analysis of TCF4 mutations causing Pitt-Hopkins syndrome shows increased seizure activity with missense mutations. Genet Med. 2009; 11(11):797-805.
Brockschmidt A, Filippi A, Charbel Issa P, et al. Neurologic and ocular phenotype in Pitt-Hopkins syndrome and a zebrafish model. Hum Genet. 2011; 130(5):645-655.
Stavropoulos D J, MacGregor D L, Yoon G. Mosaic microdeletion 18q21 as a cause of mental retardation. Eur J Med Genet. 2010; 53(6):396-399.
Xie Y-Y, Pan T-T, Xu D-E, et al. Clemastine ameliorates myelin deficits via preventing senescence of oligodendrocytes precursor cells in Alzheimer's Disease model mouse. Front Cell Dev Biol. 2021; 9:733945.
Barak B, Zhang Z, Liu Y, et al. Neuronal deletion of Gtf2i, associated with Williams syndrome, causes behavioral and myelin alterations rescuable by a remyelinating drug. Nat Neurosci. 2019; 22(5):700-708.
Deshmukh V A, Tardif V, Lyssiotis C A, et al. A regenerative approach to the treatment of multiple sclerosis. Nature. 2013; 502(7471):327-332.
Cree BAC, Niu J, Hoi K K, et al. Clemastine rescues myelination defects and promotes functional recovery in hypoxic brain injury. Brain. 2018; 141(1):85-98.
Green A J, Gelfand J M, Cree B A, et al. Clemastine fumarate as a remyelinating therapy for multiple sclerosis (ReBUILD): a randomised, controlled, double-blind, crossover trial. Lancet. 2017; 390(10111):2481-2489.
Steadman P E, Xia F, Ahmed M, et al. Disruption of oligodendrogenesis impairs memory consolidation in adult mice. Neuron. 2020; 105(1):150-164.e6.
McKenzie I A, Ohayon D, Li H, et al. Motor skill learning requires active central myelination. Science. 2014; 346(6207):318-322.
Pan Y, Cai K, Cheng M, Zou X, Li M. Responsive Social Smile: A Machine Learning based Multimodal Behavior Assessment Framework towards Early Stage Autism Screening. In: 2020 25th International Conference on Pattern Recognition (ICPR). IEEE; 2021:2240-2247.
Hartley M, Banerji T, Tagge I, et al., Myelin repair stimulated by CNS-selective thyroid hormone action. JCI Insight. 2019; 4 (8).
Barres B A, Lazar M A, Raff M C. A novel role for thyroid hormone, glucocorticoids and retinoic acid in timing oligodendrocyte development. Development. 1994; 120(5):1097-1108.
Gao F B, Apperly J, Raff M. Cell-intrinsic timers and thyroid hormone regulate the probability of cell-cycle withdrawal and differentiation of oligodendrocyte precursor cells. Dev Biol. 1998; 197(1):54-66.
Billon N, Jolicoeur C, Tokumoto Y, Vennstr6m B, Raff M. Normal timing of oligodendrocyte development depends on thyroid hormone receptor alpha 1 (TRalphal). EMBO J. 2002; 21(23):6452-6460.
Scanlan T S. Sobetirome: a case history of bench-to-clinic drug discovery and development. Heart Fail Rev. 2010; 15(2):177-182.
Ferrara S J, Meinig J M, Placzek A T, et al. Ester-to-amide rearrangement of ethanolamine-derived prodrugs of sobetirome with increased blood-brain barrier penetration. Bioorg Med Chem. 2017; 25(10):2743-2753.
Placzek A T, Ferrara S J, Hartley M D, Sanford-Crane H S, Meinig J M, Scanlan T S. Sobetirome prodrug esters with enhanced blood-brain barrier permeability. Bioorg Med Chem. 2016; 24(22):5842-5854.
Manzano J, Morte B, Scanlan T S, Bernal J. Differential effects of triiodothyronine and the thyroid hormone receptor beta-specific agonist GC-1 on thyroid hormone target genes in the brain. Endocrinology. 2003; 144(12):5480-5487.
International PCT Patent Application Publication No. WO2014178892A1 for Use of Sobetirome in the Treatment of X-Linked Drenolenoleukodystrophy, to Scanlan et al., published Nov. 6, 2014.
International PCT Patent Application Publication No. WO2016134292A1 for Derivatives of Sobetirome, to Scanlan et al., published Aug. 25, 2016.
U.S. Pat. No. 9,701,650 for Derivatives of Sobetirome, to Scanlan et al., issued Jun. 21, 2017.
U.S. Pat. No. 10,392,356 for Derivatives of Sobetirome, to Scanlan et al., issued Aug. 27, 2019.
U.S. Pat. No. 11,104,654 for Derivatives of Sobetirome, to Scanlan et al., issued Aug. 11, 2021.
U.S. Patent Application Publication No. US20190175531A1 for Sobetirome in the Treatment of Myelination Diseases, to Scanlan et al., published Jun. 13, 2019.
U.S. Patent Application Publication No. US20200405669 Alfor Sobetirome in the Treatment of Myelination Diseases, to Scanlan et al., published Dec. 31, 2020.
International PCT Patent Application Publication No. WO2018032012A1 for Amide Compounds, Pharmaceutical Compositions Thereof, and Methods of Using the Same, to Scanlan et al., published Feb. 15, 2018.
U.S. Pat. No. 11,325,886 for Amide Compounds, Pharmaceutical Compositions Thereof, and Methods of Using the Same, to Scanlan et al., issued Apr. 20, 2022.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating a disease, disorder, or condition associated with or suspected of being associated with dysmyelination, the method comprising administering to a subject in need of treatment thereof a therapeutically effective amount of sobetirome or a prodrug or derivative thereof.

2. The method of claim 1, wherein the disease, disorder, or condition associated with or suspected of being associated with dysmyelination is selected from a neurodevelopmental disorder, an intellectual disability, an autism spectrum disorder, and combinations thereof.

3. The method of claim 1, wherein the administration of sobetirome or a prodrug or derivative thereof improves or attenuates the disease, disorder, or condition associated with or suspected of being associated with dysmyelination.

4. The method of any one of claim 1, wherein the disease, disorder, or condition associated with or suspected of being associated with dysmyelination comprises Pitt-Hopkins Syndrome.

5. The method of claim 4, wherein the one or more conditions associated with Pitt-Hopkins Syndrome is selected from an intellectual disability, a developmental delay, breathing problems, recurrent seizures (epilepsy), delayed or lack of speech, impaired communication skills, impaired socialization skills, hyperventilation, apnea, cyanosis, constipation, gastrointestinal problems, microcephaly, myopia, strabismus, minor brain abnormalities, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems, and combinations thereof.

6. The method of any one of claim 1, wherein the subject has one or more single nucleotide polymorphisms in a TCF4 gene.

7. The method of any one of claim 1, wherein the subject has a chromosomal deletion including at least a portion of a TCF4 gene.

8. The method of any one of claim 1, wherein the subject has a complete deletion of a TCF4 gene.

9. The method of any one of claim 1, wherein the subject has a chromosomal translocation comprising at least a portion of a TCF4 gene.

10. The method of any one of claim 1, wherein the subject has a translocation, frameshift, or non-sense mutation in a TCF4 gene.

11. The method of any one of claim 1, wherein the subject is an infant or pediatric subject.

12. The method of any one of claim 1, wherein the subject has an age selected from about 16 years of age or less, about 12 years of age or less, about 8 years of age or less, about 5 years of age or less, and about 2 years of age or less.

13. The method of any one of claim 1, wherein the subject is an adult subject.

14. The method of any one of claim 1, wherein the administering of the sobetirome or a prodrug or derivative thereof is orally, parenterally, transdermally, sublingually, rectally, or intranasally.

15. The method of any one of claim 1, wherein the administration is by a single daily dose of sobetirome or a prodrug or derivative thereof.

16. The method of any one of claim 1, wherein the administration of sobetirome or a prodrug or derivative thereof is more than once daily.

17. The method of any one of claim 1, wherein the prodrug of sobetirome comprises Sob-AM2.

18. The method of any one of claim 1, further comprising administering one or more therapeutic agents in combination with sobetirome or a prodrug or derivative thereof.

19. The method of claim 18, wherein the one or more therapeutic agents are selected from clemastine, benzatropine, oxybutynin, trospium, ipratroprium, quetiapine, T3, XAV939, atropine, tiotropium, clobetasol, miconazole, hydroxyzine, oxiconazole, propafenone, benztropine, clotrimazole, tamoxifen, ketoconazole, dicyclomine, vesamicol, haloperidol, medroxyprogesterone, megestrol, ifenprodil, oxybutinin, bifonazole, cinanserin, betamethasone, methylprednisolone, econazole, and donepezil, or any other known or unknown pro-myelinating compound.

20. The method of claim 18, wherein the one or more therapeutic agents comprise a muscarinic receptor antagonist.

21. The method of claim 20, wherein the muscarinic receptor antagonist is selected from atropine, glycopyrronium bromide, ipratropium bromide, oxybutynin, scopolamine, tiotropium bromide, benztropine, darifenacin, fesoterodine, trihexyphenidyl, tolterodine, trospium chloride, solifenacin, propantheline bromide, and propiverine, or any other known or unknown muscarinic receptor antagonist.

22. The use of sobetirome or a prodrug or derivative thereof the manufacture of a medicament for treating a disease, disorder, or condition associated with or suspected of being associated with dysmyelination.
